(19) United States
(12) Patent Application Publication
(10) Pub. No.: US 2021/0369290 A1
(43) Pub. Date: Dec. 2, 2021

(54) NAVIGATED DRILL GUIDE

(71) Applicant: GLOBUS MEDICAL, INC., Audubon, PA (US)

(72) Inventors: Thomas Palazzolo, Bryn Mawr, PA (US); Stephen Cicchini, North Wales, PA (US); Hayden Cameron, Philadelphia, PA (US); Dana Wisniewski, Audubon, PA (US)

(21) Appl. No.: 16/883,070

(22) Filed: May 26, 2020

(51) Int. Cl. A61B 17/17; A61B 34/30; A61B 34/20

(52) U.S. Cl. CPC A61B 17/1703; A61B 17/1707; A61B 2034/2046; A61B 34/20; A61B 34/30

(57) ABSTRACT

Embodiments generally relate to preventing overpenetration of a drill bit during surgery. A system comprises a drill guide comprising a housing; a tubular member extending from the housing; a depth-stop movably disposable within the housing; and a ratchet adjacent to the depth-stop, the ratchet configured to retract or extend the depth-stop relative to the housing.

### BACKGROUND

Position recognition systems are used to determine the position of and track a particular object in 3-dimensions (3D). In robot assisted surgeries, for example, certain objects, such as surgical instruments, need to be tracked with a high degree of precision as the instrument is being positioned and moved by a robot or by a physician, for example.

Infrared signal-based position recognition systems may use passive and/or active sensors or markers for tracking the objects. In passive sensors or markers, objects to be tracked may include passive sensors, such as reflective spherical balls, which are positioned at strategic locations on the object to be tracked. Infrared transmitters transmit a signal, and the reflective spherical balls reflect the signal to aid in determining the position of the object in 3D. In active sensors or markers, the objects to be tracked include active infrared transmitters, such as light emitting diodes (LEDs), and thus generate their own infrared signals for 3D detection.

With either active or passive tracking sensors, the system then geometrically resolves the 3-dimensional position of the active and/or passive sensors. However, there are no controls to directly control depth of surgical intrusions, such as drilling with the surgical instrument, for example.

### SUMMARY

In an exemplary embodiment, the present disclosure provides A system comprises a drill guide comprising a housing; a tubular member extending from the housing; a depth-stop movably disposable within the housing; and a ratchet adjacent to the depth-stop, the ratchet configured to retract or extend the depth-stop relative to the housing.

In another exemplary embodiment, the present disclosure provides a system comprising a drill guide comprising: a housing; a tubular member extending from the housing; a depth-stop movably disposable within the housing; and a ratchet adjacent to the depth-stop, the ratchet configured to retract or extend the depth-stop relative to the housing. The system further comprises a drill assembly comprising: a tracking array comprising tracking markers; and a drill bit disposed within a portion of the tracking array; and wherein a portion of the drill assembly is disposed within the drill guide.

In another exemplary embodiment, the present disclosure provides a system comprising: a drill guide comprising: a housing; a tubular member extending from the housing; a depth-stop movably disposable within the housing; and a ratchet adjacent to the depth-stop, the ratchet configured to retract or extend the depth-stop relative to the housing. The system further comprises a drill assembly comprising: a tracking array comprising tracking markers; and a drill bit disposed within a portion of the tracking array, wherein the drill assembly is disposed within the drill guide. The system further comprises an end-effector, wherein the drill guide is disposed within the end effector.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

These drawings illustrate certain aspects of some of the embodiments of the present disclosure and should not be used to limit or define the disclosure.

FIG. 1A illustrates an exemplary embodiment of a drill assembly.

FIG. 1B illustrates a cross-section of an exemplary embodiment of the drill assembly.

FIG. 2A illustrates an exemplary embodiment of a drill guide.

FIG. 2B illustrates an exemplary embodiment a cross-section of the drill guide.

FIG. 3 illustrates an exemplary embodiment of an end-effector configured to receive the drill assembly.

FIG. 4 illustrates an exemplary embodiment of the drill guide 200 inserted concentrically into the sleeve of the end-effector.

FIG. 5 illustrates a side perspective view of an exemplary embodiment of a drilling system.

FIG. 6 illustrates an exemplary embodiment of the drill guide positioned within a tracking array.

FIG. 7 illustrates an exemplary embodiment of the drill assembly 100 with the drill bit bottomed out.

FIG. 8 illustrates an overhead view of a potential arrangement for locations of the robotic system, patient, surgeon, and other medical personnel during a surgical procedure.

FIG. 9 illustrates a robotic system including positioning of a surgical robot and a camera relative to the patient according to one embodiment.

### DETAILED DESCRIPTION

For the purposes of promoting an understanding of the principles of the present disclosure, reference will now be made to the implementations illustrated in the drawings and specific language will be used to describe them. It will nevertheless be understood that no limitation of the scope of the disclosure may be intended. Any alterations and further modifications to the described devices, instruments, methods, and any further application of the principles of the present disclosure are fully contemplated as would normally occur to one skilled in the art to which the disclosure relates. In particular, it may be fully contemplated that the features, components, and/or steps described with reference to one or more implementations may be combined with the features, components, and/or steps described with reference to other implementations of the present disclosure. For simplicity, in some instances the same reference numbers are used throughout the drawings to refer to the same or like parts.

Embodiments generally relate to spinal surgery. More particularly, embodiments relate to a drilling guide that may prevent overpenetration of a drill bit to prevent damage to critical anatomy, while maintaining an accurately navigated trajectory that may be coaxial to preplanned trajectories. The embodiments may provide: (1) trajectory guidance for the drill bit at a tip of an instrument through free hand navigation or in concert with a navigated robotic end-effector; (2) control of drill depth via a mechanical stop or depth-stop; and (3) accurate tracking of a drill trajectory while drilling via a tracked array.

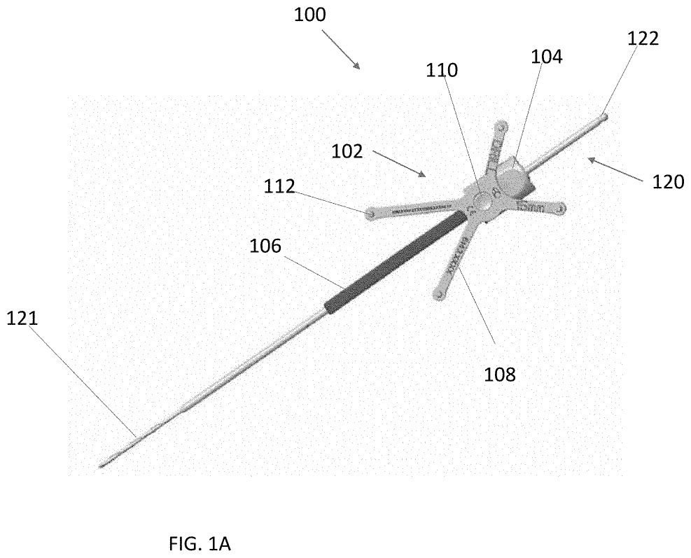

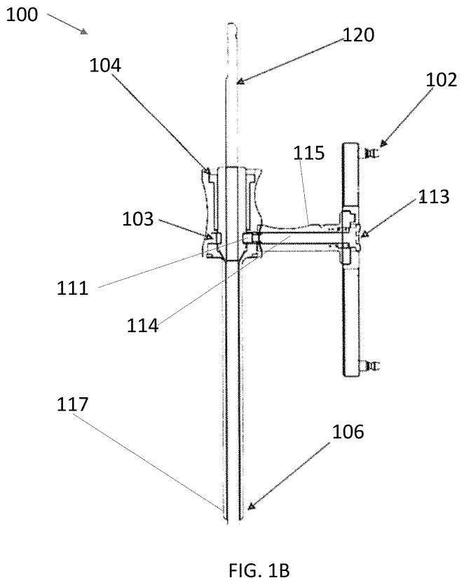

FIG. 1A illustrates an exemplary embodiment of a drill assembly 100. The drill assembly 100 may include a tracking array 102 that may include or is coupled to a tubular portion 104 configured to receive a sleeve 106 via a threaded connection, for example. The tracking array 102 may include arms 108 that extend from a central portion 110 of the tracking array 102. Distal ends of the arms 108 may be coupled to the tracking markers 112, as shown. Any suitable technique may be used for coupling the tracking array 102 to the tubular portion 104. Suitable techniques may include, but are not limited to, welds, threads, and adhesives, among others.

In some embodiments, the tubular portion 104 may be a hollow and elongated structure with an inner surface that may include threads that are configured to mate with threads positioned on an outer surface of the sleeve 106. That is, a portion (e.g., a distal end) of the sleeve 106 may be threaded (e.g., coupled or decoupled) within the tubular portion 104. At least a portion of the sleeve 106 may have an outer diameter that is less than an inner diameter of the tubular portion 104 to allow for coupling. The sleeve 106 may have an inner diameter ranging, for example, from 10 millimeters (“mm”) to 20 mm (e.g., 15 mm or 17 mm). The sleeve 106 may be removably coupled to the tubular portion 104 and may be coaxially aligned with the tubular portion 104. The sleeve 106 may be configured to receive a drill bit 120. In some examples, the sleeve 106 may serve as a bearing surface and be made of poly-ether-ether-ketone (PEEK). The sleeve 106 (and the tubular portion 104) may include a rigidity sufficient to stabilize the drill bit 120 that may be positioned and secured concentrically within the sleeve 106 and the tubular portion 104. A distal end 121 of the drill bit 120 may be configured to penetrate tissue and bone. A proximal end 122 of the drill bit 120 may include contours configured for removable attachment (e.g., press fit or twist) to a drill (not shown), such as a power drill, for example.

FIG. 1B illustrates a cross-section of an exemplary embodiment of the drill assembly 100 including the tracking array 102. The drill assembly 100 may also include a locking mechanism such as an indentation 103, for example. The indentation 103 may extend from an inner surface of the tubular portion 104. The drill bit 120 may be secured within the tubular portion 104. The drill bit 120 may be secured between a distal end 111 of a button 113 and the indentation 103, upon actuation or inward movement of the button 113. The button 113 may be or include an elongated member 114, as illustrated, for example. The button 113 may extend through a hollow shaft or member 115 that extends from the tubular portion 104. A tip 117 of the sleeve 106 may have a tolerance with respect to drill flutes (not shown) of the drill bit 120 that is sufficient to prevent excessive walk.

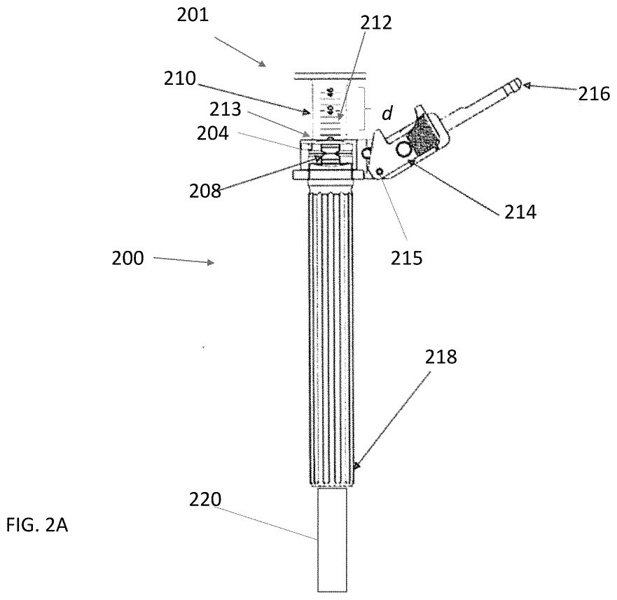

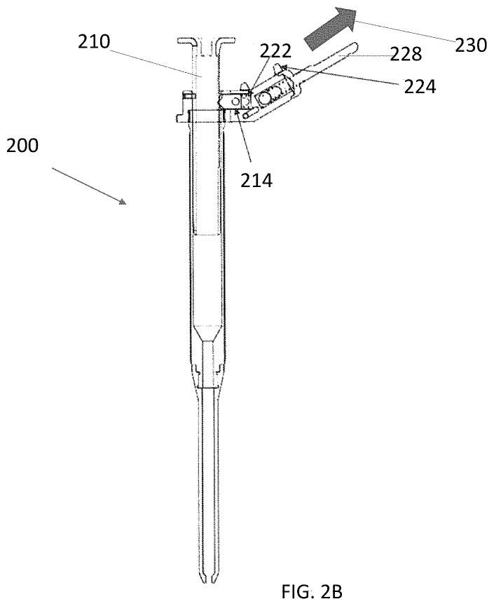

FIG. 2A illustrates an exemplary embodiment of the drill guide 200. A proximal end 201 of the drill guide 200 may include a housing 204. A depth-stop 210 may be movably disposable within the housing 204. The depth-stop 210 may be a tubular portion of the drill guide 200 that may be movably disposable within a passage 213 of the housing 204. The depth-stop 210 may be adjustable and may include notches 212 to indicate axial movement of the depth-stop 210. The housing 204 may include a depth indicator 208 that corresponds with a position of the depth-stop 210. The depth indicator 208 may include a portion or pointer that points to a notch 212 or indicates a position of the depth-stop 210.

In some embodiments, the housing 204 may include a ratchet 214. The ratchet 214 may be pivotably attached to the housing 204 via pins 215, for example. The ratchet 214 may be in contact with the a rack and thereby adjusts a position of the depth-stop 210, upon actuation of the ratchet 214. The depth-stop 210 may be ratcheted up or down. The ratchet 214 is configured to extend or retract the depth-stop 210 from the housing 204. For example, the ratchet 214 may extend or retract the depth-stop 210 a distance, d, during ratcheting adjustments. Spacing between the rack may range from 1 millimeter (“mm”) to 2 mm. Therefore, the depth-stop 210 may be adjusted in 1 to 2-mm increments. The ratchet 214 is a non-limiting example of a ratchet and other suitable ratchets may be utilized, as should be understood by one having skill in the art, with the benefit of this disclosure. The ratchet 214 may include a connection 216 for a handle (not shown). The connection 216 may include an Association for Osteosynthesis (AO) connect interface, as should be understood by one having skill in the art with the benefit of this disclosure.

The drill bit 120 (e.g., shown on FIG. 1A) may pass through the depth-stop 210 and a shaft or member 218 that may extend from the housing 204. A distal end 220 of the member 218 may be opposite to the housing 204. The member 218 may be hollow, tubular, and may be disposed in an end-effector (not shown). The depth-stop 210 prevents the drill bit 120 from exceeding a maximum drill depth determined by a position of the depth-stop 210.

FIG. 2B illustrates an exemplary embodiment a cross-section of the drill guide 200. In the illustrated embodiment, the ratchet 214 is spring loaded with a spring 222 positioned between the ratchet 214 and a lock 224. In a locked position, the spring 222 prevents translation of the ratchet 214 thereby preventing movement of the depth-stop 210. To unlock the ratchet 214, a user may pull on the lock 224 which may be located in a handle 228. The lock 224 may be moved in a direction indicated by a directional arrow 230 to decompress the spring 222 and allow translation of the ratchet 214, for example. The handle 228 may also be spring loaded (not shown) in some embodiments. After unlocking the ratchet 214, the user may adjust a position of the drill guide 200 with the ratchet 214. When the user releases the lock 224, the spring 222 moves the lock 224 back into a locked position, the ratchet 214 is secured in place and unable to translate, and the depth-stop 210 is immobilized in the chosen position. As a safety measure, a default position of the ratchet 214 is in a locked position.

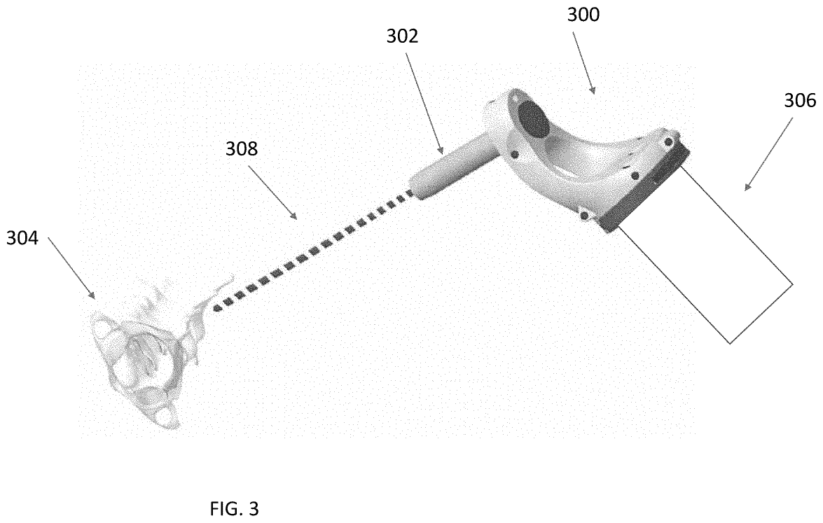

FIG. 3 illustrates an exemplary embodiment of an end-effector 300 configured to receive the drill assembly 100 (e.g., shown on FIG. 1B). The end-effector 300 may include a sleeve 302. The drill assembly 100 may be removably positioned within the sleeve 302. In some examples, the sleeve 302 verifies that a desired instrument, such as the drill (not shown), for example, is ready for navigation into an anatomical structure 304 of a human, for example. The end-effector 300 may be positioned on a distal end of a robot arm 306. As the robot arm 306 moves, a positioning of the drill and the drill assembly 100 can be monitored via the tracking markers 112 (e.g., shown on FIG. 1A). The end-effector 300 may be moved on a trajectory 308.

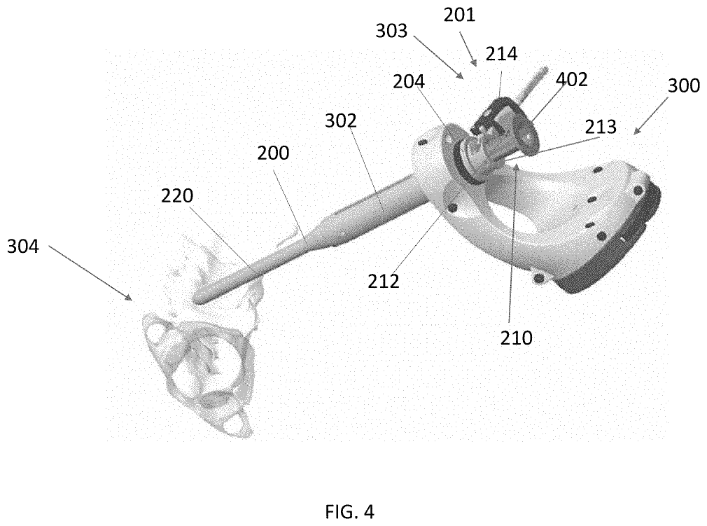

FIG. 4 illustrates an exemplary embodiment of the drill guide 200 inserted concentrically into the sleeve 302 of the end-effector 300. A distal end 220 of the drill guide 200 may extend or protrude from within the sleeve 302. The drill guide 200 may be inserted into the end-effector 300 until the distal end 220 contacts the anatomical structure 304, such as bone, for example. As noted previously, the proximal end 201 of the drill guide 200 may include the housing 204. The depth-stop 210 may be movably disposable within the passage 213 of the housing 204. The notches 212 indicate axial movement of the depth-stop 210. The ratchet 214 is configured to extends or retract the depth-stop 210 from the housing 204.

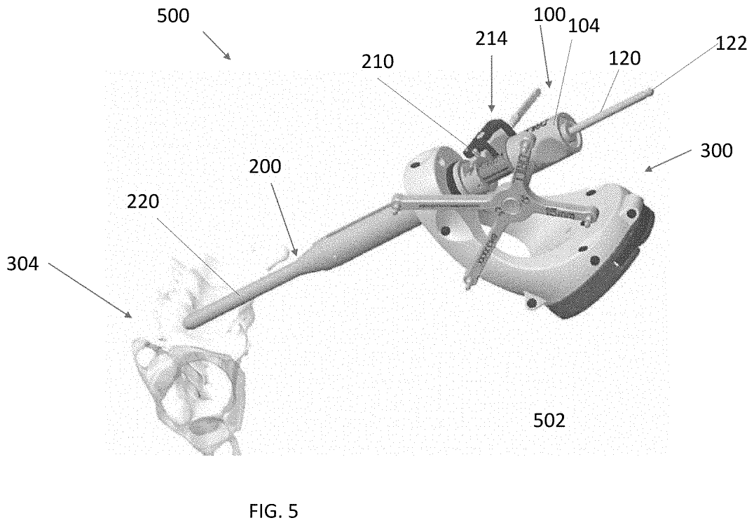

FIG. 5 illustrates a side perspective view of an exemplary embodiment of a drilling system 500. As illustrated, the drill assembly 100 is positioned within the drill guide 200. The drill guide 200 is disposed in the end-effector 300. The tubular portion 104 of the drill assembly and the depth-stop 210 of the drill guide 200 may be coaxially aligned in a stacked configuration. The drill bit 120 may be disposed within the tubular portion 104 and extend through the distal end 220 of the drill guide 200. The proximal end 122 of the drill bit 120 may be coupled to a drill (not shown). During surgery, as the drill bit 120 penetrates the anatomical structure 304, the depth-stop 210 may receive the drill bit 120 of the drill assembly 100 and prevent forward axial movement of the drill bit 120 upon contact with the tubular portion 104 of the drill assembly, to prevent overpenetration into the anatomical structure 304.

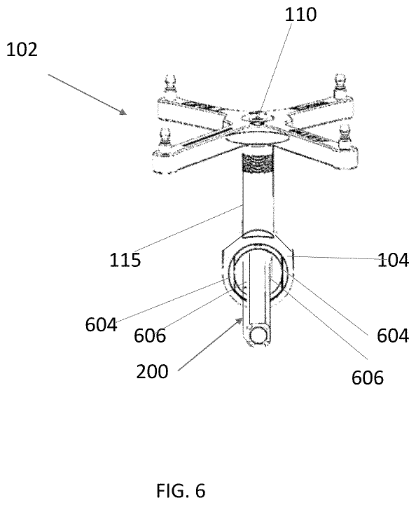

FIG. 6 illustrates an exemplary embodiment of the drill guide 200 positioned within the tubular portion 104 of the tracking array 102. The member 115 extends from the central portion 110 of the tracking array 102. The tubular portion 104 may be coupled to the member 115. The drill guide 200 may be inserted through the tubular portion 104. Internal contours such as flat portions 604 of the tubular portion 104 may correspond to external contours or flat portions 606 of the drill guide 200 to prevent independent rotation of the tracking array 102, while drilling. In certain examples, the flat portions 604 or 606 may be portions of an octagon or another shape.

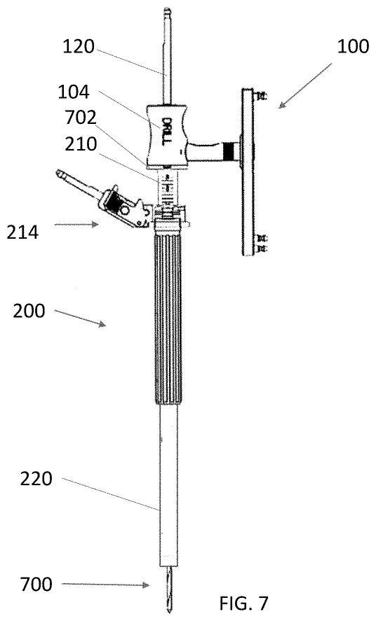

FIG. 7 illustrates an exemplary embodiment of the drill assembly 100 with the drill bit 120 bottomed out. A distal end 700 of the drill bit 120 protrudes from the distal end 220 of the drill guide 200. The drill bit 120 is secured within the tubular portion 104 of the drill guide 200. As illustrated, the tubular portion 104 is in contact with the depth-stop 210. This stacked configuration of the tubular portion 104 and the depth-stop 210 prevents further drilling or overpenetration. For example, during drilling with the drill bit 120, the tubular portion 104 eventually moves forward and contacts a flange 702 of the depth-stop 210. Upon contacting the flange 702 with the tubular portion 104, the drill bit 120 is prevented from drilling any deeper into the anatomical structure 304 (e.g., shown on FIG. 3).

With reference to FIGS. 1A-7, an exemplary technique for surgical drilling is described as follows. Locations of screws may be planned with software. A drill diameter based on a desired screw diameter may be selected. The drill bit 120 may be locked into the tubular portion 104 of the tracking array 102 of the drill assembly 100 (as shown on FIG. 1A, for example). A depth of the anatomical structure 304 to be drilled may then be determined. The depth-stop 210 may be adjusted to correspond with a desired drill depth, as shown on FIG. 2A, for example. The end-effector 300 may be aligned, via the robot arm 306, with the trajectory 308, as shown on FIG. 3, for example. Then, the drill guide 200 may be inserted into the end-effector 300 until the distal end 220 of the drill guide 200 contacts the anatomical structure 304, as shown on FIG. 4, for example. Then, the drill assembly may be inserted into the drill guide 200. Drilling may then occur until the tubular portion 104 of the drill assembly 100 bottoms out or contacts the depth-stop 210, as shown on FIG. 7, for example.

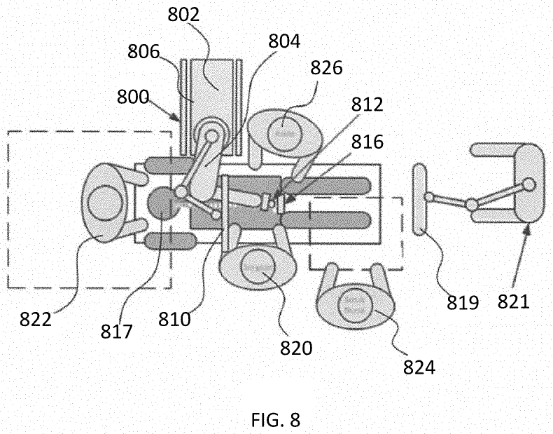

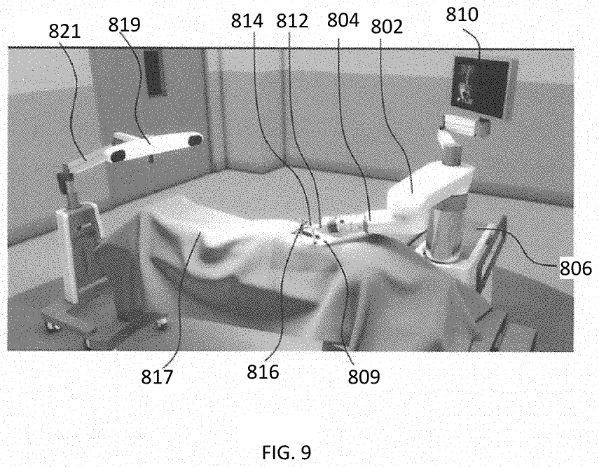

Turning now to the drawing, FIGS. 8 and 9 illustrate a surgical robot system 800 in accordance with an exemplary embodiment. Surgical robot system 800 may include, for example, a surgical robot 802, one or more robot arms 804, a base 806, a display 810, an end-effector 812, for example, including a guide tube 814, and one or more tracking markers 818 (e.g., shown on FIG. 9). The surgical robot system 800 may include a patient tracking device 816, which is adapted to be secured directly to the patient 817 (e.g., to the bone of the patient 817). The surgical robot system 800 may also utilize a camera 819, for example, positioned on a camera stand 821. The camera stand 821 can have any suitable configuration to move, orient, and support the camera 819 in a desired position. The camera 819 may include any suitable camera or cameras, such as one or more infrared cameras (e.g., bifocal or stereophotogrammetric cameras), able to identify, for example, active and passive tracking markers 818 in a given measurement volume viewable from the perspective of the camera 819. The camera 819 may scan the given measurement volume and detect the light that comes from the markers 818 in order to identify and determine the position of the markers 818 in three-dimensions. For example, active markers 818 may include infrared-emitting markers that are activated by an electrical signal (e.g., infrared light emitting diodes (LEDs)), and passive markers 818 may include retro-reflective markers that reflect infrared light (e.g., they reflect incoming IR radiation into the direction of the incoming light), for example, emitted by illuminators on the camera 819 or other suitable device.

FIGS. 8 and 9 illustrate a potential configuration for the placement of the surgical robot system 800 in an operating room environment. For example, the robot 802 may be positioned near or next to the patient 817. Although depicted near the head of the patient 817, it will be appreciated that the robot 802 can be positioned at any suitable location near the patient 817 depending on the area of the patient 817 undergoing the operation. The camera 819 may be separated from the robot system 800 and positioned at the foot of the patient 817. This location allows the camera 819 to have a direct visual line of sight to the surgical field 809 (e.g., shown on FIG. 9). Again, it is contemplated that the camera 819 may be located at any suitable position having line of sight to the surgical field 809. In the configuration shown, the surgeon 820 may be positioned across from the robot 802, but is still able to manipulate the end-effector 812 and the display 810. A surgical assistant 826 may be positioned across from the surgeon 820 again with access to both the end-effector 812 and the display 810. If desired, the locations of the surgeon 820 and the assistant 826 may be reversed. The traditional areas for the anesthesiologist 822 and the nurse or scrub tech 824 remain unimpeded by the locations of the robot 802 and camera 819.

With respect to the other components of the robot 802, the display 810 can be attached to the surgical robot 802 and in other exemplary embodiments, display 810 can be detached from surgical robot 802, either within a surgical room with the surgical robot 802, or in a remote location. End-effector 812 may be coupled to the robot arm 804 and controlled by at least one motor. In exemplary embodiments, end-effector 812 can comprise a guide tube 814, which is able to receive and orient a surgical instrument (not shown) used to perform surgery on the patient 817. As used herein, the term “end-effector” is used interchangeably with the terms “end-effectuator” and “effectuator element.” Although generally shown with a guide tube 814, it will be appreciated that the end-effector 812 may be replaced with any suitable instrumentation suitable for use in surgery. In some embodiments, end-effector 812 can comprise any known structure for effecting the movement of the surgical instrument (not shown) in a desired manner.

The surgical robot 802 is able to control the translation and orientation of the end-effector 812. The robot 802 is able to move end-effector 812 along x-, y-, and z-axes, for example. The end-effector 812 can be configured for selective rotation about one or more of the x-, y-, and z-axis, and a Z Frame axis (such that one or more of the Euler Angles (e.g., roll, pitch, and/or yaw) associated with end-effector 812 can be selectively controlled). In some exemplary embodiments, selective control of the translation and orientation of end-effector 812 can permit performance of medical procedures with significantly improved accuracy compared to conventional robots that utilize, for example, a six degree of freedom robot arm comprising only rotational axes. For example, the surgical robot system 800 may be used to operate on patient 817, and robot arm 804 can be positioned above the body of patient 817, with end-effector 812 selectively angled relative to the z-axis toward the body of patient 817.

In some exemplary embodiments, the position of the surgical instrument 608 can be dynamically updated so that surgical robot 802 can be aware of the location of the surgical instrument at all times during the procedure. Consequently, in some exemplary embodiments, surgical robot 802 can move the surgical instrument to the desired position quickly without any further assistance from a physician (unless the physician so desires). In some further embodiments, surgical robot 802 can be configured to correct the path of the surgical instrument if the surgical instrument strays from the selected, preplanned trajectory. In some exemplary embodiments, surgical robot 802 can be configured to permit stoppage, modification, and/or manual control of the movement of end-effector 812 and/or the surgical instrument. Thus, in use, in exemplary embodiments, a physician or other user can operate the system 800, and has the option to stop, modify, or manually control the autonomous movement of end-effector 812 and/or the surgical instrument.

The robotic surgical system 800 can comprise one or more tracking markers 818 configured to track the movement of robot arm 804, end-effector 812, patient 817, and/or the surgical instrument in three dimensions. In exemplary embodiments, a plurality of tracking markers 818 can be mounted (or otherwise secured) thereon to an outer surface of the robot 802, such as, for example and without limitation, on base 806 of robot 802, on robot arm 804, or on the end-effector 812. In exemplary embodiments, at least one tracking marker 818 of the plurality of tracking markers 818 can be mounted or otherwise secured to the end-effector 812. One or more tracking markers 818 can further be mounted (or otherwise secured) to the patient 817. In exemplary embodiments, the plurality of tracking markers 818 can be positioned on the patient 817 spaced apart from the surgical field 809 to reduce the likelihood of being obscured by the surgeon, surgical tools, or other parts of the robot 802. Further, one or more tracking markers 818 can be further mounted (or otherwise secured) to the surgical tools (e.g., a screw driver, dilator, implant inserter, or the like). Thus, the tracking markers 818 enable each of the marked objects (e.g., the end-effector 812, the patient 817, and the surgical tools) to be tracked by the robot 802. In exemplary embodiments, system 800 can use tracking information collected from each of the marked objects to calculate the orientation and location, for example, of the end-effector 812, the surgical instrument (e.g., positioned in the tube 814 of the end-effector 812), and the relative position of the patient 817.

The markers 818 may include radiopaque or optical markers. The markers 818 may be suitably shaped include spherical, spheroid, cylindrical, cube, cuboid, or the like. In exemplary embodiments, one or more of markers 818 may be optical markers. In some embodiments, the positioning of one or more tracking markers 818 on end-effector 812 can maximize the accuracy of the positional measurements by serving to check or verify the position of end-effector 812. Further details of surgical robot system 800 including the control, movement and tracking of surgical robot 802 and of a surgical instrument can be found in U.S. patent application Ser. No. 13/924,505, which is incorporated herein by reference in its entirety.

Exemplary embodiments include one or more markers 818 coupled to the surgical instrument. In exemplary embodiments, these markers 818, for example, coupled to the patient 817 and surgical instruments, as well as markers 818 coupled to the end-effector 812 of the robot 802 can comprise conventional infrared light-emitting diodes (LEDs) or an Optotrak® diode capable of being tracked using a commercially available infrared optical tracking system such as Optotrak®. Optotrak® is a registered trademark of Northern Digital Inc., Waterloo, Ontario, Canada. In other embodiments, markers 818 can comprise conventional reflective spheres capable of being tracked using a commercially available optical tracking system such as Polaris Spectra. Polaris Spectra is also a registered trademark of Northern Digital, Inc. In an exemplary embodiment, the markers 818 coupled to the end-effector 812 are active markers which comprise infrared light-emitting diodes which may be turned on and off, and the markers 818 coupled to the patient 817 and the surgical instruments comprise passive reflective spheres.

In exemplary embodiments, light emitted from and/or reflected by markers 818 can be detected by camera 819 and can be used to monitor the location and movement of the marked objects. In alternative embodiments, markers 818 can comprise a radio-frequency and/or electromagnetic reflector or transceiver and the camera 819 can include or be replaced by a radio-frequency and/or electromagnetic transceiver.

The present disclosure, as described above, describes many features which allow improved control and precision of a surgical drilling operation. For example, the drill guide ensures that a maximum drill depth is controlled with a mechanical or hard stop to prevent overpenetration with a drill bit. A drill trajectory may be controlled via an end-effector of a robot arm for improved control. Also, a drill position may be tracked during surgery with the tracking array for improved accuracy.

It is believed that the operation and construction of the present disclosure will be apparent from the foregoing description. While the apparatus and methods shown or described above have been characterized as being preferred, various changes and modifications may be made therein without departing from the spirit and scope of the disclosure as defined in the following claims.

What is claimed is:

1. A drill guide comprising:
a housing;
a tubular member extending from the housing;
a depth-stop movably disposable within the housing; and
a ratchet adjacent to the depth-stop, the ratchet configured to retract or extend the depth-stop relative to the housing.

2. The drill guide of claim 1, wherein the depth-stop includes notches.

3. The drill guide of claim 2, wherein the notches are spaced apart in 1 to 2 millimeter increments.

4. The drill guide of claim 1, further comprising a lock configured to prevent translation of the ratchet.

5. The drill guide of claim 4, wherein the lock is adjacent to a spring.

6. The drill guide of claim 1, wherein the depth-stop includes a passage configured to receive the drill bit.

7. The drill guide of claim 1, wherein the ratchet is pivotably attached to the housing.

8. A system comprising:
a drill guide comprising:
a housing;
a tubular member extending from the housing;
a depth-stop movably disposable within the housing; and
a ratchet adjacent to the depth-stop, the ratchet configured to retract or extend the depth-stop relative to the housing; and
a drill assembly comprising:
a tracking array comprising tracking markers; and
a drill bit disposed within a portion of the tracking array; and
wherein a portion of the drill assembly is disposed within the drill guide.

9. The system of claim 8, wherein the depth-stop includes notches.

10. The system of claim 9, wherein the notches are spaced apart in 1 to 2 millimeter increments.

11. The system of claim 10, wherein the drill guide further comprises a lock configured to prevent translation of the ratchet.

12. The system of claim 11, wherein the lock is adjacent to a spring.

13. The system of claim 8, wherein the depth-stop includes a passage configured to receive the drill bit.

14. The system of claim 8, wherein the ratchet is pivotably attached to the housing.

15. A system comprising:
a drill guide comprising:
a housing;
a tubular member extending from the housing;
a depth-stop movably disposable within the housing; and
a ratchet adjacent to the depth-stop, the ratchet configured to retract or extend the depth-stop relative to the housing; and
a drill assembly comprising:
a tracking array comprising tracking markers; and
a drill bit disposed within a portion of the tracking array, wherein a portion of the drill assembly is disposed within the drill guide; and
an end-effector, wherein the drill guide is disposed within the end-effector.

16. The system of claim 15, further comprising a robot arm coupled to the end-effector.

17. The system of claim 15, wherein the depth-stop includes notches.

18. The system of claim 17, wherein the notches are spaced apart in 1 to 2 millimeter increments.

19. The system of claim 15, wherein the drill guide further comprises a lock configured to prevent translation of the ratchet.

20. The system of claim 19, wherein the lock is adjacent to a spring.
